(19) United States
(12) Patent Application Publication
(10) Pub. No.: US 2016/0183874 A1
(43) Pub. Date: Jun. 30, 2016

(54) ATTACHMENT DEVICE FOR ATTACHING AN ELECTRONIC DEVICE

(71) Applicant: KABUSHIKI KAISHA TOSHIBA, Tokyo (JP)

(72) Inventors: Minoru TAKIZAWA, Sagamihara Kanagawa (JP); Yasushi SASAKI, Mitaka Tokyo (JP); Hidetaka HASHIMOTO, Ome Tokyo (JP)

(21) Appl. No.: 14/700,475

(22) Filed: Apr. 30, 2015

(30) Foreign Application Priority Data
Dec. 26, 2014 (JP) 2014-266299

(51) Int. Cl. A61B 5/00; A61B 5/0408

(52) U.S. Cl. CPC A61B 5/6832; A61B 5/0408

(57) ABSTRACT

An attachment device for attaching an electronic device on a body of a subject includes an attachment member and a battery. The attachment member has a first surface to be in contact with the electronic device and a second surface that is opposite to the first surface and formed of an adhesive material configured to adhere to the body of the subject. The battery has a terminal to be electrically connected with the electronic device to supply power to the electronic device.

### CROSS-REFERENCE TO RELATED APPLICATION

This application is based upon and claims the benefit of priority from Japanese Patent Application No. 2014-266299, filed Dec. 26, 2014, the entire contents of which are incorporated herein by reference.

### FIELD

Embodiments described herein relate generally to an attachment device for attaching an electronic device on a body of a person.

### BACKGROUND

In the related art, an electronic apparatus (e.g., a medical device) operates in a state of being attached to a body of a person.

### DESCRIPTION OF THE DRAWINGS

FIG. 1 is a perspective view of an attachment device according to a first embodiment and an electronic apparatus to which the attachment device is attached.

FIG. 2 is a perspective view of the attachment device according to the first embodiment.

FIG. 3 is a perspective view illustrating an attachment of the attachment device according to the first embodiment to the electronic apparatus.

FIG. 4 is a plan view of the attachment device and the electronic apparatus, that illustrates power supply from a battery on the attachment device to the electronic apparatus.

FIG. 5 is a plan view of an attachment device according to a second embodiment and an electronic apparatus to be coupled therewith.

FIG. 6 is a plan view of an attachment device according to a third embodiment and an electronic apparatus to be coupled therewith.

FIG. 7 is a plan view of the attachment device, explaining an attached state and a detached state of a battery.

FIG. 8 is a plan view of an attachment device according to a fourth embodiment.

FIG. 9 illustrates a configuration of a battery fixed to an attachment member of the attachment device according to the fourth embodiment.

FIG. 10 illustrates another configuration of the battery fixed to the attachment member.

FIG. 11 illustrates a configuration of an attachment device according to a fifth embodiment.

FIG. 12 is a perspective view of the attachment device according to the fifth embodiment that is mounted on an electronic apparatus.

FIG. 13 illustrates a usage example of an electronic apparatus according to a sixth embodiment.

### DETAILED DESCRIPTION

One or more exemplary embodiments are directed to provide a compact electronic apparatus that can operate for a predetermined drive time while being attached to a body surface of a user.

In general, according to one embodiment, an attachment device for attaching an electronic device on a body of a subject includes an attachment member and a battery. The attachment member has a first surface to be in contact with the electronic device and a second surface that is opposite to the first surface and formed of an adhesive material configured to adhere to the body of the subject. The battery has a terminal to be electrically connected with the electronic device to supply power to the electronic device.

The same configuration elements are included in a plurality of embodiments or modification examples in the following description. Thus, hereinafter, the same symbols or reference numerals will be used for the same configuration elements, and repeated description thereof will be omitted.

### First Embodiment

An attachment device 10 (gel sheet, adhesive pad, gel pad) according to a first embodiment has a flexibility, and has a flat shape, for example, a shape of a rectangular sheet. The attachment device 10 is attachable to a surface 14a (sensor surface, top surface, first wall) of a housing 14 of an electronic apparatus 12.

The electronic apparatus 12 is a portable sensor unit which can detect, for example, a cardiac potential or the like. The housing 14 of the electronic apparatus 12 is formed by insert molding using a synthetic resin material, and is formed in such a manner that a substrate which supports a plurality of electronic components or electrodes (described below) is covered with a synthetic resin material that is inserted at the time of insert molding. The housing 14 has, for example, a flat cuboid shape, and electrodes 16a and 16b (probes, terminals, metals, conductors) are disposed on the surface 14a, in a state in which detection surfaces (sensor surfaces, end portions, surfaces, one-end surfaces) thereof are exposed on the surface 14a. The electrode 16a is, for example, a “positive electrode,” the electrode 16b is, for example, a “negative electrode,” and both are disposed separately from each other. When the electronic apparatus 12 detects a biological signal (potential, cardiac potential) for generating an electrocardiogram, if a distance between the electrode 16a and the electrode 16b is equal to or greater than a predetermined distance, a more stable detection result may be obtained. Meanwhile, the smaller the electronic apparatus 12 is, the better portability and availability of the electronic apparatus 12 are. Therefore, in the embodiment, the electrode 16a and the electrode 16b are diagonally disposed on the surface 14a, and thereby a size of the electronic apparatus 12 can be reduced, while a predetermined distance is secured between the electrode 16a and the electrode 16b.

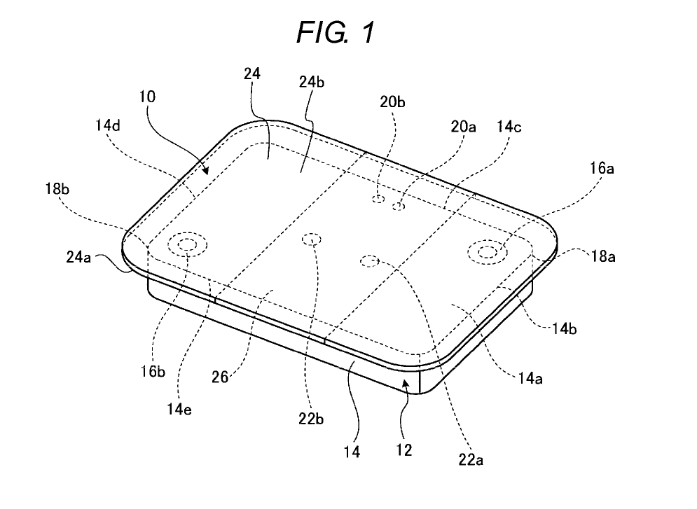

In FIG. 1, the electrode 16a is disposed in a position close to a corner 18a at which a side 14b and a side 14c of the housing 14 intersect. Meanwhile, the electrode 16b is disposed in a position close to a corner 18b at which a side 14d and a side 14e intersect. In this way, the electrode 16a and the electrode 16b are diagonally disposed, whereby it is possible to secure a distance between the electrode 16a and the electrode 16b without increasing the size of the housing 14, for example, compared to a case in which the electrode 16a and the electrode 16b are arranged in a direction parallel to the side 14c or in a direction parallel to the side 14b.

In addition, the housing 14 is bendable and has flexibility (softness). For example, the housing 14 is made of a soft resin with flexibility. Then, it is possible for the housing 14 to be bent such that a bus is formed in a direction intersecting the sides 14c and 14e, which extend in a longitudinal direction of the housing 14. The electrode 16a is disposed in a position close to the corner 18a, that is, on one end in a longitudinal direction of the housing 14, and the electrode 16b is disposed in a position close to the corner 18b, that is, on the other end in a longitudinal direction of the housing 14. As a result, when the electronic apparatus 12 is in contact with a body surface (i.e., a curved surface), it is possible to increase adhesion of the electrode 16a and the electrode 16b, which are arranged at end positions in a longitudinal direction, to the body surface, as the housing 14 is curved in accordance with the body surface.

In addition, data input and output terminals 20a and 20b (connectors, contact points, electrodes, metals, conductors) are disposed and exposed on the surface 14a. The input and output terminals 20a and 20b may be used for transmission of data, for example, when a detected value, data, information based on the detected value, or the like that is acquired by the electronic apparatus 12 is transmitted to an external apparatus using a wire method, or when updating of software for controlling the electronic apparatus 12 is performed using a wire method or the like. The input and output terminals 20a and 20b may be electrically connected to a terminal of an adaptor apparatus, such as a cradle or the like.

In FIG. 1, the input and output terminals 20a and 20b are arranged substantially in parallel to the side 14c, in a position close to, for example, the side 14c. The input and output terminals 20a and 20b are not used to detect a biological signal. In addition, a current does not flow between the input and output terminal 20a and the input and output terminal 20b. Thus, it is not necessary to dispose the input and output terminals 20a and 20b separately from each other in the same manner as the electrodes 16a and 16b, and the input and output terminals 20a and 20b may be located close to each other.

The location of the electrodes 16a and 16b and the input and output terminals 20a and 20b is an example, and the position of each electrode or terminal may be different according to a specification of the electronic apparatus 12.

In addition, on the surface 14a, conductors 22a and 22b (first power terminals, connectors, contact points, electrodes, metals) are exposed in an area in which other electrodes or terminals do not exist, for example, in a central area. The conductors 22a and 22b are terminals for receiving a power from an external power supply unit. The conductor 22a is, for example, a “+ power supply terminal,” the conductor 22b is, for example, a “− power supply terminal,” and are both disposed so as to be separated by a predetermined distance from each other.

A biological signal, which is detected through the electrodes 16a and 16b, is stored in a storage unit mounted on a substrate of the electronic apparatus 12, and is transmitted to an external apparatus such as an output device (electrocardiograph, monitor device, printing device) of an electrocardiogram at a desired timing, or is transferred to a personal computer, a server, or the like. In addition, the biological signal may be directly transmitted to the output device of the electrocardiogram or a portable terminal through the electrodes 16a and 16b. The electronic apparatus 12 according to the embodiment may transmit the biological signal or the like to an external apparatus using a wire method through the input and output terminals 20a and 20b. In addition, it is possible to transmit the biological signal to an external apparatus via a communication unit such as Bluetooth (registered trademark). In this case, for example, it is possible to monitor an electrocardiogram of a subject for 24 hours. It is possible to transmit data in a predetermined interval, to transfer data at a desired timing, or to update software of the electronic apparatus 12, via a communication unit such as Bluetooth.

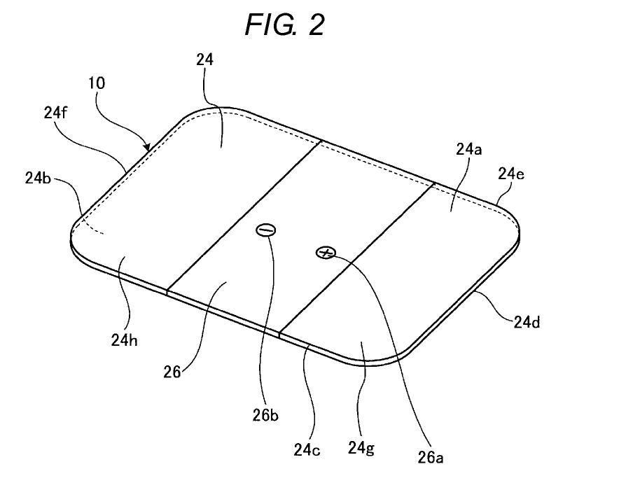

As illustrated in FIGS. 1 and 2, the attachment device 10 includes an attachment member 24 (adhesive layer, base, retaining layer) with an adhesive, and a battery 26 (battery cell, cell, battery layer). The attachment member 24 may be attached to the surface 14a of the housing 14 of the electronic apparatus 12. In order to increase adhesion of the electrode 16a and the electrode 16b of the electronic apparatus 12 to a body surface, the attachment device 10 is disposed between the surface 14a and a body surface. As the attachment device 10 has conductivity, it is possible to detect a cardiac potential of a user (subject) at the electrode 16a and the electrode 16b through the attachment device 10.

The attachment member 24 has a first surface 24a (attachment surface, power supplying surface, first wall, surface), at least a portion of the electronic apparatus 12 which includes the conductors 22a and 22b, and which is attached to the electronic apparatus 12, and a second surface 24b (attachment surface, contact surface, second wall, rear surface) which is positioned in an opposite side to the first surface 24a, has adhesion, and is attached to a user. It is desirable that the first surface 24a of the attachment member 24 is fixed to the surface 14a of the electronic apparatus 12. For example, the first surface 24a may be fixed to the surface 14a with adhesive property of the attachment member 24. Alternatively, the first surface 24a may be fixed to the surface 14a of the electronic apparatus 12 with mechanical connection between a fixing member extending from the attachment member 24, (e.g., a belt-shaped member), and the electronic apparatus 12. Further alternatively, the first surface 24a of the attachment member 24 and the electronic apparatus 12 may each have portions which fit to each other, and the first surface 24a may be fixed to the surface 14a by fitting the portions. Meanwhile, the second surface 24b of the attachment member 24 has an adhesive strength for attaching the electronic apparatus 12 to a body surface of a user via the attachment member 24. In the embodiment, the first surface 24a of the attachment member 24 is attached to the surface 14a of the electronic apparatus 12 with adhesive property of the attachment member 24, and the adhesion can be maintained.

The battery 26 is fixed to the attachment member 24, and at least a portion of the battery 26 is covered with the electronic apparatus 12. The battery 26 includes electrode 26a and 26b (second power terminals, connectors, contact points, terminals, metals, conductors) that are electrically connected to the conductors 22a and 22b, respectively, and supply power to the electronic apparatus 12, when the attachment member 24 is attached to the electronic apparatus 12. For example, the battery 26 may be a primary battery, which has a sheet shape and is bendable in correspondence to flexibility of the attachment member 24. The battery 26 may be fixed on the first surface 24a of the attachment member 24 by the adhesive property of the attachment member 24, and may be inserted and fixed in an internal layer of the attachment member 24.

The electrodes 26a and 26b are located in the substantially center position of an attachment member 24, in which a center line extending in a longitudinal direction of the attachment member 24 interests a center extending in a lateral direction. The distance between the electrodes 26a and 26b corresponds to a distance between the conductors 22a and 22b. For example, the electrode 26a is a “positive electrode,” and the electrode 26b is a “negative electrode.” It is necessary for the electrode 16a and the electrode 16b, which are used to detect a cardiac potential, to be electrically insulated from each other. For this reason, by dividing the attachment member 24 into a first gel area 24g on the electrode 16a and a second gel area 24h on the electrode 16b by the battery 26, the electrode 16a and the electrode 16b may be electrically insulated from each other. In addition, instead of by the battery 26, the electrode 16a and the electrode 16b may be electrically insulated by an insulating material.

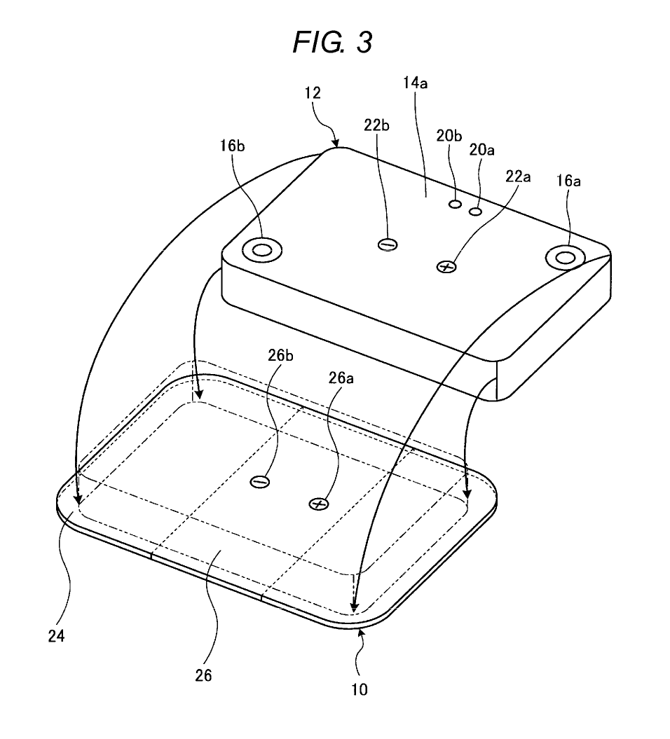

According to the attachment device 10 configured in this way, it is possible to supply a power from the battery 26 to the electronic apparatus 12, by stacking the electrode 26a on the conductor 22a so as to be electrically connected to each other, and by stacking the electrode 26b on the conductor 22b so as to be electrically connected to each other, as illustrated in FIG. 3.

A structure for coupling the attachment device 10 with the electronic apparatus 12 at a predetermined position may be provided on the surface 14a of the electronic apparatus 12 and the first surface 24a of the attachment member 24, so that a connection of the electrode 26a and the conductor 22a and a connection of the electrode 26b and the conductor 22b can be easily performed. For example, the conductors 22a and 22b may slightly protrude from the surface 14a. Meanwhile, the electrodes 26a and 26b may be recessed from the first surface 24a by an amount corresponding to an amount of protrusion of the conductors 22a and 22b. With this configuration and a combined effect of a concave portion and a convex portion, it is possible to easily and correctly couple the electrode 26a with the conductor 22a and the electrode 26b with the conductor 22b. In addition, as the electrodes 26a and 26b are recessed from the first surface 24a of the attachment member 24 in this way, it is possible to suppress unintentional electrical connection between the electrode 26a and the electrode 26b, when the attachment device 10 is not attached to the electronic apparatus 12.

As a modification example, a fitting structure different from the electrodes 26a and 26b and the conductors 22a and 22b may be provided. For example, when the battery 26 is fixed on the first surface 24a of the attachment member 24, the entire battery 26 may protrude from the first surface 24a. At this time, a concave portion corresponding to a shape of the battery 26 may be provided on the surface 14a of the electronic apparatus 12, whereby the entire battery 26 may be fit in the concave portion, and electrical connection between the electrode 26a and the conductor 22a, and between the electrode 26b and the conductor 22b may be easily and correctly made. In addition, as the battery 26 is fit into the concave portion, offset between the attachment member 24 and the electronic apparatus 12 is unlikely to happen, and electrical connection of between the electrode 26a and the conductor 22a and between the electrode 26b and the conductor 22b can be maintained, after the attachment member 24 is attached to the electronic apparatus 12.

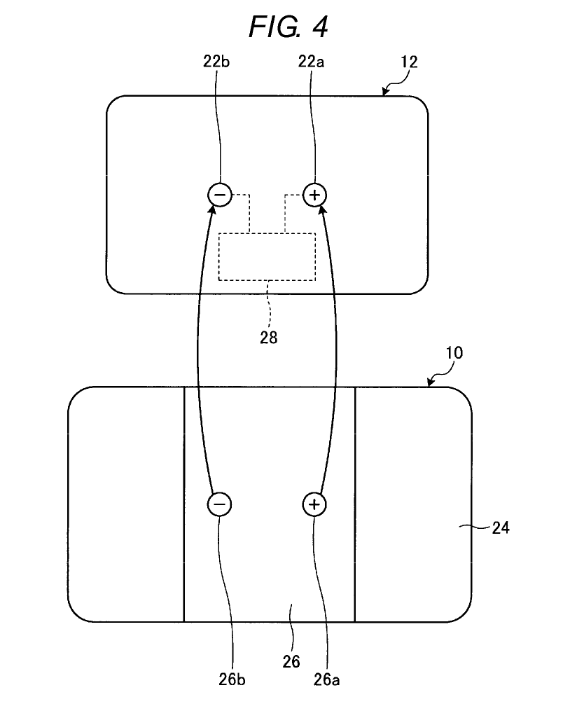

As illustrated in FIG. 4, by attaching the attachment device 10 to the electronic apparatus 12, it is possible to supply power from the battery 26 fixed to the attachment member 24 to a power supply circuit 28 included in the electronic apparatus 12. That is, it is not necessary for the electronic apparatus 12 to include a battery for driving the power supply circuit 28, and miniaturization and weight reduction of the electronic apparatus 12 is possible. In addition, it is possible to dispose other electronic components in a space in which a battery would be disposed inside the electronic apparatus 12, to reduce a size of the electronic apparatus 12 and to add a new function to the electronic apparatus 12. As a result, a higher performance of the electronic apparatus 12 may be achieved.

Capacity of the battery 26 may be determined according to a continuous drive time of the electronic apparatus 12. For example, when a continuous detection (e.g., 24 hours) of a cardiac potential is performed, it is desirable that a sufficient capacity of the battery 26 is provided in the attachment device 10.

In addition, since the battery 26 has a sheet shape and is bendable according to the shape of the attachment member 24, the battery 26 is easily bent along a curved body surface of a subject when being attached to the body surface of the subject. As a result, the subject may not feel uncomfortable even if the electronic apparatus 12 is used for a long period of time. In addition, since it is possible to easily maintain adhesion of the electronic apparatus 12 to the body surface, it is possible to stably acquire a biological signal such as a cardiac potential.

The battery 26 fixed to the attachment member 24 of the attachment device 10 according to the first embodiment is a primary battery which performs only discharge. Thus, when an output of the battery 26 is less than a predetermined value, the attachment device 10 is disposed of together with the battery 26. In addition, after being separated from each other, each of the attachment member 24 and the battery 26 can be separately disposed of.

### Second Embodiment

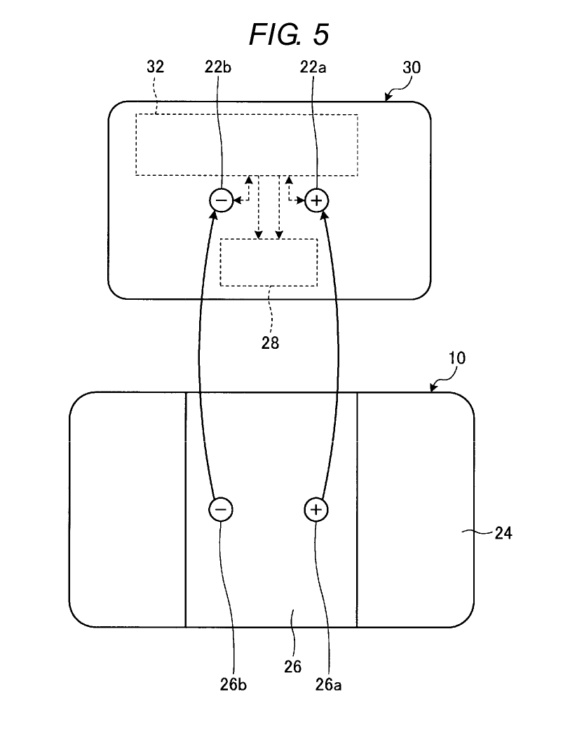

FIG. 5 is a plan view of the attachment device 10 and an electronic apparatus 30 according to a second embodiment. The electronic apparatus 12 according to the first embodiment does not include an internal battery, but a secondary battery 32 (a first secondary battery) is included in the inside of the electronic apparatus 30 according to the second embodiment. In this case, the battery 26 on the attachment device 10 may function as an auxiliary battery with respect to the electronic apparatus 30. For example, the electronic apparatus 30 includes a power supply circuit 28 operated with a power from a secondary battery 32, and performs a detection operation of a cardiac potential or the like, a storing operation of the detected cardiac potential, or the like.

When an output of the secondary battery 32 is equal to or higher than a predetermined value, even if the electrodes 26a and 26b are electrically connected to the conductors 22a and 22b, power is not supplied from the battery 26 to the electronic apparatus 30 (secondary battery 32). Meanwhile, when the output of the secondary battery 32 is less than a predetermined value, the electronic apparatus 30 receives a power from the battery 26 attached to the attachment device 10 via the secondary battery 32, supply power to the power supply circuit 28, and continues to perform a detection operation of a cardiac potential or the like, the storing operation of the detected cardiac potential, or the like. That is, by using the battery 26 as an auxiliary battery, it is possible to perform a long-time operation of the electronic apparatus 30. Alternatively, in another embodiment, drive of the electronic apparatus 12 may be initially performed by power supplied from the battery 26, and when the output of the battery 26 is less than a predetermined value, the secondary battery 32 may supply power for driving the electronic apparatus 30.

In this way, power for driving the electronic apparatus 30 is supplied from the secondary battery 32 and the battery 26, whereby it is possible to share battery capacity. That is, compared to a case in which the same long-time drive is performed only by the electronic apparatus 30, miniaturization or weight reduction of the electronic apparatus 30 is possible. In addition, when the same long-time drive is performed by the electronic apparatus 12 according to the first embodiment, it is necessary for the attachment device 10 to include the battery with large capacity. Meanwhile, according to the configuration of the second embodiment, the secondary battery 32 and the battery 26 may provide a required capacity together, and thus miniaturization and a weight decrease of each battery are possible.

In addition, the secondary battery 32 may be used first for a standard drive time of the electronic apparatus 30, and the battery 26 may be used subsequently for an additional drive time of the electronic apparatus 30. For example, if a plurality of attachment devices 10 includes batteries 26 with different capacities, it is possible to drive the electronic apparatus 30 for different periods of time depending on the capacity of the battery 26 in the attachment device 10. As a result, it is possible to increase usability of the electronic apparatus 30. When a power is supplied from the battery 26 to the electronic apparatus 30, the power may be supplied directly to the power supply circuit 28 not passing through the secondary battery 32.

### Third Embodiment

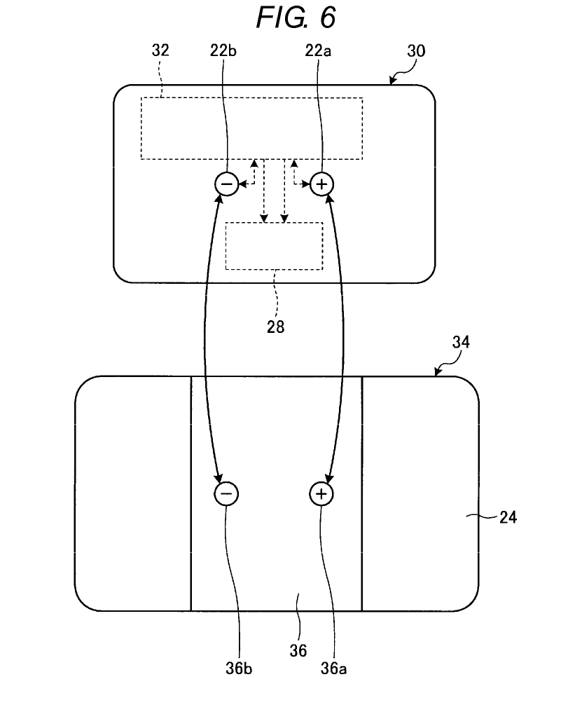

FIG. 6 is an explanatory view of an attachment device and an electronic apparatus 30 according to a third embodiment. Since a configuration of the electronic apparatus 30 is the same as the apparatus according to the second embodiment, description thereof will be omitted.

The battery 26 attached to the attachment device 10 according to the first embodiment and the second embodiment is a primary battery which performs only a discharge, but the attachment member 24 of the attachment device 34 according to the third embodiment includes a secondary battery 36 (second secondary battery) which is rechargeable.

In the same manner as the second embodiment, also in the third embodiment, the secondary battery 36 on the attachment device 34 may function as an auxiliary battery with respect to the electronic apparatus 30. That is, when an output of the secondary battery 32 attached to the electronic apparatus 30 is equal to or greater than a predetermined value, power is not supplied to the electronic apparatus 30 (secondary battery 32) from the secondary battery 36, even when electrodes 36a and 36b are electrically connected to the conductors 22a and 22b. By contrast, when the output of the secondary battery 32 is less than a predetermined value, the electronic apparatus receives power form the secondary battery 36 on the attachment device 34 via the secondary battery 32, supplies power to the power supply circuit 28, and continues to perform a detection operation such as a cardiac potential, the storing operation of the detected cardiac potential, or the like. That is, it is possible to perform a long-time operation of the electronic apparatus 30 by using the secondary battery 36 as an auxiliary battery.

Alternatively, as another embodiment, the secondary battery 32 may be used as an auxiliary battery, and the secondary battery 36 may be used as a main battery for driving the electronic apparatus 30. As a result, also in the configuration according to the third embodiment, it is possible to obtain the same effects as in the second embodiment.

Furthermore, since the secondary battery 36 of the attachment device 34 is rechargeable, the secondary battery 36 may be recharged by the power from the secondary battery 32 in the electronic apparatus 30. In the same manner, when the electronic apparatus 30 is connected to a cradle or the like and electrically connected to an external power supply, the secondary battery 36 can be recharged. That is, when the electronic apparatus 30 is not operated, the secondary battery 36 may be recharged.

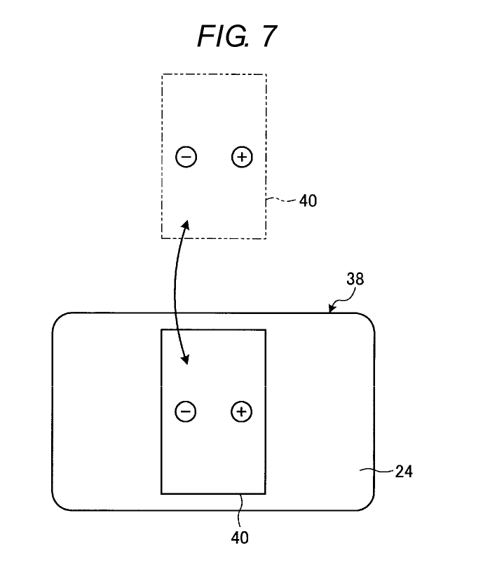

Here, when a battery 40 (primary battery or secondary battery) is attachable to and detachable from the attachment member 24 in the same manner as an attachment device 38 illustrated in FIG. 7, the battery 40 may be used again after recharging. For example, when the electronic apparatus 30 is attached to a body surface of a subject with an adhesive property of the attachment member 24 for a long time, the adhesive strength of the attachment member 24 may decrease by sweat or foreign materials. At this time, it is possible to separate the battery 40 from the attachment member 24 and to replace the attachment member 24 with a new one (one with sufficient adhesive strength). Then, by attaching the charged battery 40 to the new attachment member 24, an attachment device 38 may be used again. That is, by exchanging only the attachment member 24, a user may use the attachment device 38 which has a sufficient adhesive strength, is clean, and moreover can supply a sufficient power, at a low cost.

When the battery 40 is attached to the attachment member 24, the battery 40 may be attached with an adhesive property of the attachment member 24, or may include another structure for the attachment. For example, the battery 40 may be fixed using a double-sided tape, a hook and loop fastener, or the like. In addition, when the battery 40 is inserted in an inner layer portion of the attachment member 24, the attachment member 24 may have a multi-layer structure. For example, a portion of a surface layer of the attachment member 24 may be formed so as to be openable, and the battery 40 may be contained within the attachment member 24. In this case, the surface layer is covered from above, and the battery 40 may be housed within the attachment member 24.

In addition, when the battery 40 is a primary battery, by providing an attachable and detachable structure as described above, the attachment member 24 and the battery 40 can be separated from each other. As a result, they are able to be disposed separately, and it may promote recycling.

### Fourth Embodiment

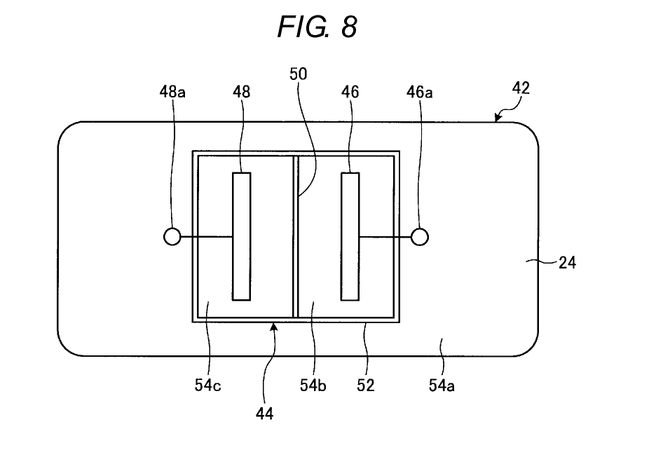

FIG. 8 is a perspective view of an attachment device 42 according to a fourth embodiment. A portion or the whole of the attachment member 24 includes a gel layer which includes, for example, sodium chloride and water as a main ingredient, and thus the gel layer may include electrolyte, while adhesive property or flexibility of an attachment device is maintained. That is, a battery 44 may function with a component of the attachment device 42.

In FIG. 8, the attachment member 24 includes a gel layer which includes, for example, sodium chloride and water as a main ingredient, and a cathode electrode 46, an anode electrode 48, and a separator 50 are disposed in an inner layer portion (in gel layer) of the attachment member 24. The separator 50 includes non-woven fabric, a porous thin film, or the like, which is disposed between the cathode electrode 46 and the anode electrode 48, and the cathode electrode 46 and the anode electrode 48 are electrically separated. In addition, the cathode electrode 46, the anode electrode 48, and the separator 50 are surrounded by a partition wall 52, which is made of non-woven fabric, a porous thin film, or the like, and insulates an inside and an outside of the battery 44, in the gel layer of the attachment member 24. As described above, the attachment member 24 is also in contact with the electrode 16a and the electrode 16b for detecting a cardiac potential, which are included in the electronic apparatus 12 (30), and thus a current of the battery 44 does not affect the detection of the cardiac potential.

An electrode 46a (connector, contact point, terminal, metal, conductor) extends from the cathode electrode 46, and an electrode 48a (connector, contact point, terminal, metal, conductor) extends from the anode electrode 48. Thus, both of the electrode 46a and the electrode 48a may be electrically connected to the conductors 22a and 22b of the electronic apparatus 12 (30). The attachment member 24 includes a first area 54a, which is an outer side (region in which cathode electrode 46 and anode electrode 48 are not included) than the partition wall 52, a second area 54b, which is in an internal region of the partition wall 52 including the cathode electrode 46, and a third area 54c, which is in an internal region of the partition wall 52 including the anode electrode 48. In the battery 44, if the second area 54b and the third area 54c are electrolytes, ions may move between the cathode electrode 46 and the anode electrode 48, and charging or discharging may be performed. Thus, it is not necessary for the first area 54a to be an electrolyte.

In the present embodiment, the first area 54a, the second area 54b, and the third area 54c include gel layers which include sodium chloride and water as a main ingredient. That is, in the battery 44, the cathode electrode 46, the anode electrode 48, the separator 50, the partition wall 52, and the like are embedded in the attachment member 24 formed of one sheet. In this way, by configuring the entire attachment member 24 using a gel layer of an electrolyte, it is possible to simplify the configuration of the electronic apparatus and to reduce manufacturing cost. A type of metals which configure the cathode electrode 46 and the anode electrode 48, or ingredients of the gel layer which configures the attachment member 24, are appropriately selected according to a format of a battery. As an example, as the battery terminals, the cathode electrode 46 (positive electrode) may be made from copper and the anode electrode 48 (negative electrode) may be made from zinc.

In this way, when in the battery 44, the attachment member 24 includes a gel layer with electrolyte and the cathode electrode 46 and the anode electrode 48 are disposed in the gel layer, the battery 44 may not likely to decrease flexibility of the attachment member 24, compared to a structure in which an independent battery is fixed to the attachment member 24. As a result, it is possible to provide the soft attachment device 42 which is easily bent and easily adheres to a surface of a body or the electronic apparatus 12 (30).

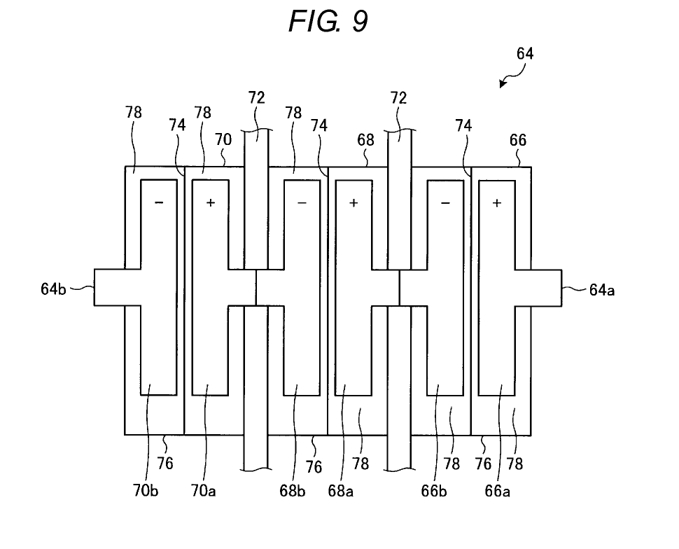

FIG. 9 schematically illustrates a battery 64 supported by the attachment member 24 (FIG. 2). In FIG. 9, in order to increase an output voltage of the battery 64, a plurality of battery cells 66 to 70 (e.g., three battery cells) is connected in series. Then, an insulating materials 72 (insulating sheets) are disposed between the battery cell 66 and the battery cell 68, and between the battery cell 68 and the battery cell 70, whereby the battery cells are electrically insulated from each other. In addition, a separator 74 is disposed between a cathode electrode 66a and an anode electrode 66b, which are included in the battery cell 66, and the cathode electrode 66a and the anode electrode 66b are not in direct contact with each other. In the same manner, a cathode electrode 68a and an anode electrode 68b of the battery cell 68, and a cathode electrode 70a and an anode electrode 70b of the battery cell 70 are respectively separated by the separators 74. Each of the battery cells 66 to 70 is surrounded by an insulating member 76, such that an electrolyte 78 contained in the battery cells does not leak.

The anode electrode 66b of the battery cell 66 is electrically connected to the cathode electrode 68a of the battery cell 68, and the anode electrode 68b of the battery cell 68 is electrically connected to the cathode electrode 70a of the battery cell 70. That is, the battery cells 66 to 70 are connected in series, a terminal 64a extending from the cathode electrode 66a and a terminal 64b extending from the anode electrode 70b respectively serve as terminals of the battery 64, and are configured so as to be able to be electrically connected to the conductors 22a and 22b of the electronic apparatus 12 (30). The battery 64 is bendable along a longitudinal direction of, for example, the insulating material 72, whereby the battery cells 66 to 70 may be folded. That is, each battery cell may be stacked in a state of being connected in series, may be folded in a compact manner, and may be supported by the attachment member 24 (refer to FIG. 2). As a result, it is possible to increase the output voltage without increasing a size (sheet area) of the attachment device 10 (34, 38, 42).

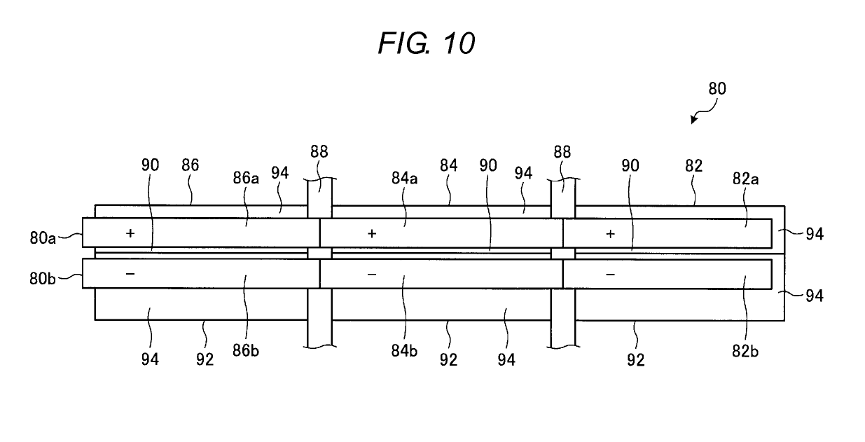

FIG. 10 illustrates a battery 80 which is supported by the attachment member 24 (refer to FIG. 2). FIG. 10 illustrates a case in which, in order to increase capacity of the battery 80, a plurality of battery cells 82 to 86 (three battery cells as an example, in FIG. 10) are connected in parallel. Then, insulating materials 88 (insulating sheet) are disposed between the battery cell 82 and the battery cell 84 and between the battery cell 84 and the battery cell 86, and the battery cells are insulated from each other. In addition, a separator 90 is disposed between a cathode electrode 82a and an anode electrode 82b of the battery cell 82, and the cathode electrode 82a and the anode electrode 82b are not in direct contact with each other. In the same manner, a cathode electrode 84a and an anode electrode 84b of the battery cell 84, and a cathode electrode 86a and an anode electrode 86b of the battery cell 86 are respectively separated by the separator 90. Each of the battery cells 82 to 86 is surrounded by an insulating member 92, such that an electrolyte 94 contained in the battery cells does not leak.

The cathode electrodes 82a, 84a, and 86a are electrically connected in series, the anode electrodes 82b, 84b, and 86b are electrically connected in series, and as the entire battery 80, the battery cell 82, the battery cell 84, and the battery cell 86 are electrically connected in parallel. A terminal 80a extending from the cathode electrode 86a and a terminal 80b extending from the anode electrode 86b respectively serve as terminals of the battery 80, and are configured so as to be able to be electrically connected to the conductors 22a and 22b of the electronic apparatus 12 (30). The battery 80 is bendable along a longitudinal direction of, for example, the insulating material 88, whereby the battery cells 82 to 86 may be folded. That is, each battery cell may be stacked in a state of being connected in parallel, may be folded in a compact manner, and may be supported by the attachment member 24 (FIG. 2). As a result, it is possible to increase battery capacity without increasing a size (sheet area) of the attachment device 10 (34, 38, 42). In FIG. 9 and FIG. 10, the number of battery cells that are connected in series or in parallel may be appropriately determined according to the output voltage or the battery capacity that are necessary for the attachment device 10 (34, 38, 42). However, when the battery cell is a cell for a secondary battery, it is preferable that the number of battery cells to be connected is determined based on a charging and discharging cycle or an amount of charging and discharging.

### Fifth Embodiment

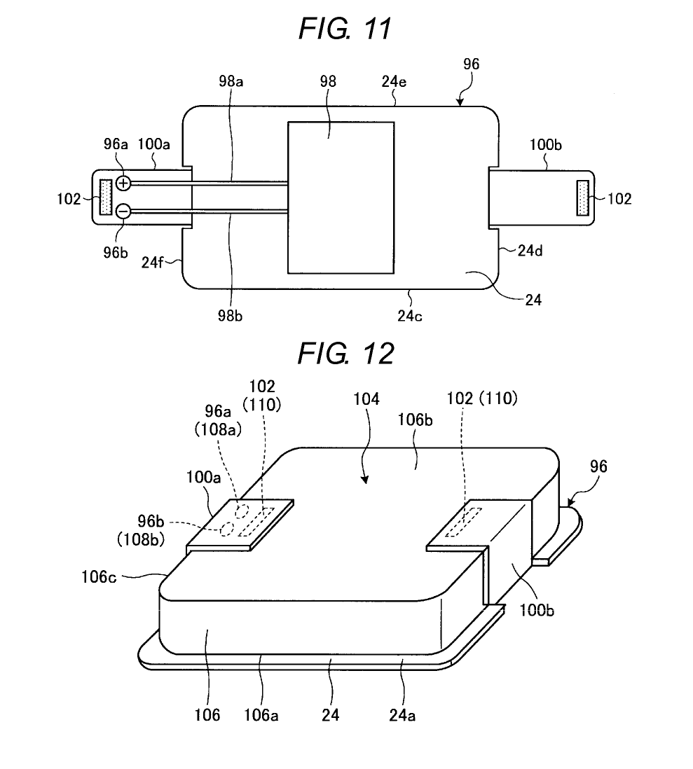

FIG. 11 illustrates an attachment device 96 according to a fifth embodiment. In the same manner as the other embodiments, a battery 98 is attached to the attachment member 24 of the attachment device 96. However, in the battery 98, differently from the battery 26 of the attachment device 10 illustrated in FIG. 2, electrodes 96a and 96b for discharging or charging are disposed in a position separated from the battery 98 and connected to the battery through lead wires 98a and 98b. In the attachment device 96 illustrated in FIG. 11, a connection member 100a (connecting piece, winding piece, fixing piece) that has flexibility and is formed in a strip shape extends in a direction along which a side 24c (24e) extends, from the substantially central portion of a side 24f in a lateral direction of the attachment member 24.

The electrodes 96a and 96b are arranged in a direction parallel to the side 24f and on the connection member 100a, and separated with a predetermined interval. In the same manner, a connection member 100b (connecting piece, winding piece, fixing piece) having a strip shape in the same manner as the connection member 100a, is extends in a direction along which the side 24c (24e) extends, from the substantially central portion of a side 24d of the attachment member 24. In FIG. 11, the electrodes 96a and 96b are not disposed on the connection member 100b, but the electrode 96a may be disposed on the connection member 100a and the electrode 96b may be disposed on the connection member 100b. In addition, the connection members 100a and 100b each include a fixing member 102 such as hook and loop fastener on sides farther from the side 24f (24d).

A configuration of an electronic apparatus 104 to which the attachment device 96 is attached is illustrated in FIG. 12. A basic configuration of the electronic apparatus 104 is the same as that of the electronic apparatus 12 (30) illustrated in the other embodiments, but positions of conductors 108a and 108b, which are exposed on a surface of a housing 106 so as to be connected to the battery 98 of the attachment device 96, are different from the electronic apparatus 12. In the electronic apparatus 12 illustrated in FIG. 1, the conductors 22a and 22b are disposed in a substantially central portion of the surface 14a of the housing 14, which is in contact with the first surface 24a of the attachment member 24 of the attachment device 10. Meanwhile, in the electronic apparatus 104, the conductors 108a and 108b are disposed on a rear surface 106b, which is opposite to a surface 106a of the housing 106 that is in contact with the first surface 24a of the attachment member 24 of the attachment device 96.

Furthermore, the conductors 108a and 108b are disposed in positions close to a side surface 106c of the housing 106. That is, as illustrated in FIG. 12, in a state in which the surface 106a (sensor surface, top surface, first wall) of the housing 106 adheres to the first surface 24a of the attachment device 96 (attachment member 24), the connection members 100a and 100b are wound so as to be in contact with the rear surface 106b of the housing 106. That is, the conductors 108a and 108b are disposed so as to be in electrical contact with the electrodes 96a and 96b disposed on the connection member 100a. In addition, on the rear surface 106b of the housing 106, a fixing member 110 (for example, a hook and loop fastener) is disposed in a position where the fixing member 110 is in mechanical contact with the fixing member 102 provided on the connection members 100a and 100b.

When the attachment device 96 configured as described above is attached to the electronic apparatus 104, the first surface 24a of the attachment member 24 (i.e., attachment device 96) first adheres to the surface 106a of the housing 106. At this time, unlike the attachment device 10 and the electronic apparatus 12 illustrated in FIG. 1, in order to electrically connect the electrodes 26a and 26b (refer to FIG. 2) to the conductors 22a and 22b, respectively, it is not necessary to precisely adjust a mounting position. Thereafter, the connection member 100a wraps around the rear surface 106b so as to be wound around the housing 106, and thus the electrode 96a is electrically connected to the conductor 108a, and the electrode 96b is electrically connected to the conductor 108b.

Here, since the connection member 100a is a strip shaped member with flexibility, it is possible to easily perform a position correction using flexibility of the connection member 100a, even if an attached position of the attachment device 96 with respect to the electronic apparatus 104 is slightly shifted. As a result, it is possible for the electrode 96a to be easily and electrically connected to the conductor 108a, and for the electrode 96b to be easily and electrically connected to the conductor 108b. In addition, at the time of the winding operation, a connection (for example, a hook and loop fastener) of the fixing member 102 on the attachment device 96 and the fixing member 110 on the electronic apparatus 104 is performed, and the attachment device 96 is fixed to the electronic apparatus 104. In the same manner, the connection member 100b is wrapped around the rear surface 106b so as to be wound around the housing 106, the fixing member 102 on the attachment device 96 and the fixing member 110 on the electronic apparatus 104 are coupled (for example, with a hook and loop fastener), and the attachment device 96 is fixed to the electronic apparatus 104.

In this way, the attachment device 96 is attached to the housing 106 of the electronic apparatus 104 by the adhesive property of the attachment member 24 and a fixing of the connection members 100a and 100b wound around the housing 106, and thus it is possible to maintain a stable mounting state (adhesion state of attachment device 96 and electronic apparatus 104) of the attachment device 96 and the electronic apparatus 104 for a long period of time. As a result, it is possible to perform a stable detection of a biological signal by the electronic apparatus 104. In addition, as illustrated in FIG. 12, since the conductors 108a and 108b are disposed on a surface of the housing 106 different from a surface that is supposed to be in contact with a body surface of a subject, the user (subject) may not worry about being contact with the conductors 108a and 108b. In FIG. 12, the conductors 108a and 108b are disposed on the rear surface 106b of the housing 106, but it is possible to obtain the same effects, as long as the conductors 108a and 108b are disposed on a surface other than the surface 106a.

In addition, in FIG. 11 and FIG. 12, the connection members 100a and 100b are wound to the end portions of the rear surface 106b of the housing 106, but the connection members 100a and 100b may be wound entirely around the rear surface 106b. For example, the connection member 100a and the connection member 100b may overlap each other on the rear surface 106b, and may completely enclose the housing 106. In this case, adhesive strength of the attachment device 96 with respect to the electronic apparatus 104 may be further increased. In addition, as fixing by winding is performed using the connection members 100a and 100b, shifting or missing of the electronic apparatus 104 with respect to the attachment device 96 may be further suppressed.

### Sixth Embodiment

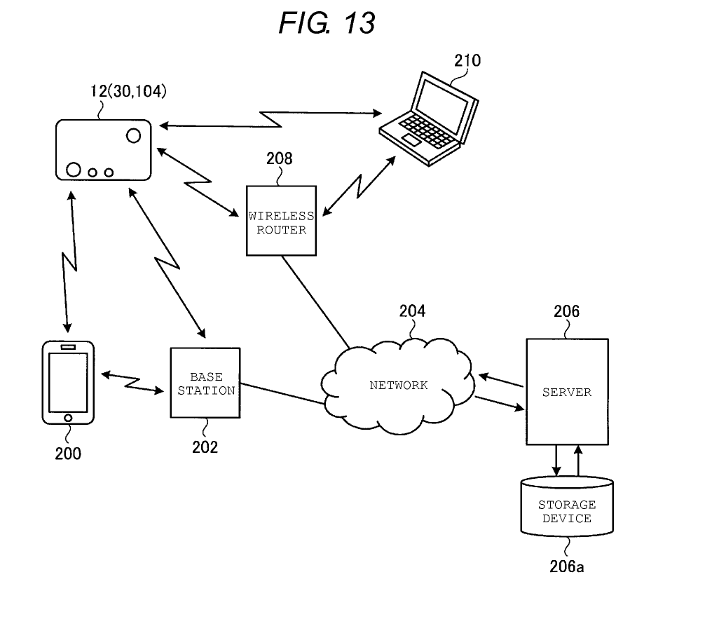

A usage example of the electronic apparatus 12 (30, 104) according to the above-described embodiments will be described using FIG. 13. When the electronic apparatus 12 detects a biological signal (potential, cardiac potential, detected value) for an electrocardiogram, the electronic apparatus 12 transmits biological information (information, transmission information), which is obtained based on the detected biological signal, to an external apparatus. For example, the electronic apparatus 12 transfers the biological information (information, transmission information) to a communication terminal 200 (mobile phone, smart phone) being carried by a user through an embedded communication function, such as Bluetooth. Here, the communication terminal 200 may transmit the acquired biological information to a server 206, which is an external apparatus, through a base station 202 or a network 204. Alternatively, the electronic apparatus 12 may transmit the detected biological signal as it is towards the server 206.

In addition, when the electronic apparatus 12 includes a connection function to the network 204 such as a Wi-Fi communication function, the electronic apparatus 12 may transmit the biological information (biological signal) to the server 206 through the base station 202 and the network 204. When the electronic apparatus 12 is connectable to a wireless LAN, the electronic apparatus 12 may transmit the biological information to the server 206 through a wireless router 208 and the network 204. The electronic apparatus 12 may transmit the biological information through the wireless router 208 via a personal computer 210.

In the above-describe example, a communication network (electrical communication circuit) using wireless is described, but a communication network using a wire may be used. The communication network includes, for example, a router, a modem, an access point, a cable, and the like. In addition, each apparatus may transmit and receive data according to a predetermined communication protocol.

Each time the electronic apparatus 12 acquires the biological information, the electronic apparatus 12 may transmit the acquired information to the server 206. Alternatively, the electronic apparatus 12 may transmit the information after a predetermined amount of signal is accumulated. Further alternatively, the electronic apparatus 12 may transmit the information every predetermined time period, and may transmit the information at a desired timing of a user in accordance with an operation of the electronic apparatus 12.

When transmitting the biological information to the server 206, the electronic apparatus 12 may transmit the biological information together with personal ID and password which are provided to each user, such that each user may be identified by the server 206. It is also possible to transmit the information without specifying an individual person, and using a guest ID.

When the server 206 acquires biological information, the server 206 stores the biological information in a storage device 206a, and performs processing according to the biological information. For example, when the biological information indicates a cardiac potential, the server 206 generates an electrocardiogram. Furthermore, the server 206 performs analysis of the electrocardiogram and generates health condition information based on the analysis. In addition, when the biological information indicates a pulse wave signal or a temperature signal, the server 206 converts the signal into a pulse or body temperature, and generates the health condition information based on the pulse or the body temperature.

When the server 206 generates the health condition information, the server 206 generates an electrocardiogram based the biological information obtained during a predetermined time period, and generates a graph of a pulse or body temperature. In addition, the server 206 may generate diagnostic information based on the generated data. In addition, when the user continually transmits the biological information to the server 206 using a personal ID, the server 206 may perform diagnosis of a long-term health condition based on a comparison of a past analysis result or diagnostic information and a newest analysis result or diagnostic information, and may generate future advice or the like as health information.

The server 206 stores the generated health diagnosis information in the storage device 206a, and returns the health diagnosis information to the user who sent the biological information through the network 204.

For example, when the user transmits the biological information through the communication terminal 200, the health diagnosis information is displayed on a display screen of the communication terminal 200.

When the user directly transmits the biological information to the server 206, using the communication function of the electronic apparatus 12, the server 206 transmits the health diagnosis information to the electronic apparatus 12. In response to receiving the health diagnosis information, the electronic apparatus 12 transfers the health diagnosis information to the communication terminal 200 or the personal computer 210 that a user owns, and the health diagnosis information is displayed on a display screen of the communication terminal 200 or the personal computer 210.

In the same manner, when the electronic apparatus 12 transmits the biological information to the server 206 through the wireless router 208, the health diagnosis information may be transmitted to the personal computer 210 of the user, and the health diagnosis information may be displayed on the display screen of the personal computer 210 of the user.

The health diagnosis information transmitted from the server 206 may be stored in the communication terminal 200 or the personal computer 210. The biological signal detected by the electronic apparatus 12 may be stored in the communication terminal 200 or the personal computer 210 as original data.

In the present embodiment, biological information based on a biological signal detected by the electronic apparatus 12 is transmitted to server 206 and is analyzed by the server 206. As another embodiment, a dedicated program may be installed in the communication terminal 200 or the personal computer 210, and the electrocardiogram or the health diagnosis information may be generated by the communication terminal 200 or the personal computer 210. In addition, a simple analysis or simple health diagnosis information may be generated by the communication terminal 200 or the personal computer 210, and more detailed analysis or health diagnosis information may be generated by the server 206 according to a request of a user.

As described above, the attachment device according to one or more embodiments includes an attachment member having a first surface to be in contact with the electronic device and a second surface that is opposite to the first surface and formed of an adhesive material that is adhesive to the body surface of the subject, and a battery having a terminal to be electrically connected with the electronic device for power supply to the electronic device. According to this configuration, it may be not necessary to include a battery inside the electronic apparatus, and it is possible to secure a predetermined drive time, while reducing a size and a weight of the electronic apparatus. In addition, by selecting capacity of the battery of the attachment device, a user may easily select a drive time of the electronic apparatus.

In addition, the battery of the attachment device may charge a first secondary battery provided in the electronic apparatus. According to this configuration, the battery on the attachment device may function as an auxiliary battery with respect to the electronic apparatus. As a result, a plurality of batteries may drive the electronic apparatus, and a long time drive of the electronic apparatus may be achieved.

In addition, the battery of the attachment device may be a second secondary battery. According to this configuration, only the attachment member may be exchanged, and the battery may be used again. As a result, running cost of the electronic apparatus, which uses the attachment device, may be reduced.

In addition, the attachment member of the attachment device may include a gel layer with an electrolyte, and the battery may include the gel layer, and an anode, a separator, and a cathode which are disposed in the gel layer. According to this configuration, the battery is unlikely to cause a decrease of flexibility of the attachment member, compared to a case where an independent battery is fixed to the attachment member.

In addition, the battery of the attachment device may be attachable and detachable to the attachment member. According to this configuration, it is easy to separate the battery from the attachment member, and to perform discarding work. In addition, it is easy to use the battery again.

In addition, the battery of the attachment device may include a connection member which electrically connects the power terminal provided in a surface different from a surface facing the first surface. According to this configuration, the user can easily recognize a connection position of a power terminal of the electronic apparatus and the battery, and to perform connection work. In addition, since the power terminal is disposed on a side different from a body surface of the user, the user may not feel uncomfortable.

In addition, the connection member of the attachment device may be wound around a housing of the electronic apparatus. According to this configuration, for example, the attachment device may be mounted on the electronic apparatus with adhesive property of the attachment member and a fixing property of the connection member, and thus it is possible to maintain a stable mounting state for a long period of time.

In each embodiment described above, the electronic apparatus is a sensor unit for detecting a cardiac potential. Alternatively, the electronic apparatus may be any electronic apparatus which needs power supply the attachment device to be attached to a subject. For example, the electronic apparatus may be a heart rate monitor, a pulsimeter, or a low frequency therapy apparatus which does not detect a biological signal and conversely gives an electrical stimulation, or the like. In such cases, as well, it is possible to obtain the same effects as in each embodiment described above.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An attachment device for attaching an electronic device on a body of a subject, comprising:
an attachment member having a first surface to be in contact with the electronic device and a second surface that is opposite to the first surface and formed of an adhesive material configured to adhere to the body of the subject; and
a battery having a terminal to be electrically connected with the electronic device to supply power to the electronic device.

2. The attachment device according to claim 1, wherein the battery is a rechargeable battery.

3. The attachment device according to claim 1, wherein
the battery is detachable from the attachment member.

4. The attachment device according to claim 1, wherein
the attachment member includes a gel layer on the second surface containing electrolytes, and
components of the battery are included in the gel layer.

5. The attachment device according to claim 4, wherein
the gel layer includes a first region and a second region that are separated from each other, and
an anode of the battery is included in the first region, and a cathode of the battery is included in the second region.

6. The attachment device according to claim 1, wherein
the battery includes a plurality of sub-battery units connected in series.

7. The attachment device according to claim 1, wherein
the battery includes a plurality of sub-battery units connected in parallel.

8. The attachment device according to claim 1, wherein
the terminal is exposed on the first surface of the attachment member.

9. The attachment device according to claim 1, wherein
the attachment member includes a first portion on which a first main surface of the electronic device is to be placed and a second portion that extends from the first portion.

10. The attachment device according to claim 9, wherein
the terminal is disposed on a surface of the second portion.

11. The attachment device according to claim 10, wherein
the terminal is disposed in a position to be connected to a terminal on a second main surface of the electronic device that is opposite to the first main surface.

12. An apparatus to be attached to a body of a subject, comprising:
an electronic device unit having an electrode to be electrically connected with the subject, on a main surface thereof, and a power terminal;
a battery unit having a terminal electrically connected with the power terminal; and
an attachment member having a first surface that is in contact with the main surface of the electronic device unit and a second surface that is opposite to the first surface and formed of an adhesive material configured to adhere to the body of the subject.

13. The apparatus according to claim 12, wherein
the electronic device unit includes an inner battery that is chargeable by power from the battery.

14. The apparatus according to claim 12, wherein
the battery and the attachment member are detachable from the electronic device unit.

15. The apparatus according to claim 12, wherein
the electronic device unit is configured to detect a biological signal of the subject.

16. The apparatus according to claim 12, wherein
the electronic device unit is configured to generate an electrical pulse to be applied to the subject.

17. The apparatus according to claim 12, wherein
the attachment member includes a first portion that is in contact with the main surface of the electronic device unit and a second portion that extends from the first portion.

18. The apparatus according to claim 17, wherein
the terminal is disposed on a surface of the second portion.

19. The apparatus according to claim 18, wherein
the terminal is connected to a terminal disposed on a second main surface of the electronic device unit that is opposite to the main surface.

20. The apparatus according to claim 19, wherein
the electronic device unit has a first coupling unit on the second main surface thereof, and
the second portion of the attachment member includes a second coupling unit that is coupled with the first coupling unit.
